(19) United States
(12) Patent Application Publication
(10) Pub. No.: US 2008/0274551 A1
(43) Pub. Date: Nov. 6, 2008

(54) Reagent, system and method for nitrate analysis

(76) Inventor: Craig R. Chinchilla, Glen Ellyn, IL (US)

Correspondence Address: RYNDAK & SURI LLP, 200 W. MADISON STREET, SUITE 2100, CHICAGO, IL, 60606, US

(21) Appl. No.: 11/799,416

(22) Filed: May 1, 2007

(51) Int. Cl. G01N 33/00; G01N 35/10

(52) U.S. Cl. 436/2; 422/63; 422/67

(57) ABSTRACT

A reagent and colorimetric autoanalyzer are provided for determining nitrate concentration using an EPA approved method. Methods of determining nitrate concentration using a reagent and a calorimetric autoanalyzer are also provided. The device and use thereof allows for the automated and accurate determination of nitrate in a sample, resulting in a non-hazardous method that is not labor intensive to determine the amount of nitrate in the sample.

### FIELD OF THE INVENTION

The present invention relates to a colorimetric autoanalyzer and the reagent and method utilized in the calorimetric autoanalyzer used for nitrate analysis.

### BACKGROUND OF THE INVENTION

Often times, it is necessary and desirable to determine the nitrate concentration of a sample. Some methods for determining nitrate concentration are not approved methods by the US Environmental Protection Agency (EPA). However, the methods that are approved by the EPA utilize hazardous chemicals, potentially expose users, and generate toxic waste, or are not automated, forcing individuals to separately run each sample, and therefore, are extremely laborious and time-consuming.

In view of the foregoing, there exists a need for an autoanalyzer that can handle and analyze a plurality of samples without requiring individual handling of each sample. Additionally, there exists a need for a reagent and method for nitrate analysis that can be used in an autoanalyzer, allowing for an efficient and quick process of analyzing a plurality of samples for the concentration and amount of nitrate in the samples.

### SUMMARY OF THE INVENTION

In accordance with the present invention, reagents and systems for nitrate analysis and methods of nitrate analysis are provided. The reagents, systems and methods are particularly useful for quick non-hazardous and automated analysis of the amount of nitrate in a sample, without requiring the user to process each individual sample. The system allows for quick non-hazardous automated nitrate analysis that can meet EPA approval requirements. The invention provides a dependable, reliable, accurate and fast nitrate analysis that can be used in an automated system.

In one aspect of the invention, a calorimetric autoanalyzer for determining nitrate concentration is provided. The calorimetric autoanalyzer comprises a plurality of sample positions for holding sample containers; a plurality of reagent containers for holding a reagent comprising effective amounts of hydrochloric acid, vanadium trichloride, sulfanilamide, and N-1-naphthylethylenediamine for nitrate analysis; a plurality of sample positions; at least one sample dispensing arm to automatically transfer sample from the sample containers to a reaction compartment; at least one reagent dispensing arm to automatically transfer reagent from the reagent containers to the reaction compartment; and a spectrophotometer for analyzing nitrate concentration, wherein the colorimetric autoanalyzer provides substantially real-time determination of sample results.

The calorimetric autoanalyzer may contain as many sample positions and sample containers as desired. Typically, the autoanalyzer comprises approximately 30-70 sample positions. More typically, the autoanalyzer comprises approximately 60 sample positions. The calorimetric autoanalyzer may contain as many reagent containers as desired. Typically, the autoanalyzer comprises approximately 35 reagent containers.

The calorimetric autoanalyzer typically is controlled by an external computer through a bi-directional communication link. Typically, the bi-directional communication link is a bi-directional serial link or is a bi-directional USB link. The autoanalyzer typically provides substantially real-time monitoring of reagent volumes, system solutions, and waste levels. Additionally, the autoanalyzer typically provides substantially real-time monitoring of sample status and sample results.

In another aspect of the invention, a method of determining nitrate concentration using an automated calorimetric autoanalyzer and a quick non-hazardous automated nitrate analysis method that can meet EPA approval requirements and reagent is provided. A colorimetric autoanalyzer is provided. At least one sample containing nitrate is added to a sample container in the colorimetric autoanalyzer. A reagent is added to a reagent container in the autoanalyzer. The reagent comprises effective amounts of hydrochloric acid, vanadium trichloride, sulfanilamide, and N-1-naphthylethylenediamine for nitrate analysis. The sample and the reagent are mixed in the autoanalyzer, thereafter, allowing the sample to react with the reagent sufficiently to permit the determination of the amount of nitrate in the sample. The reaction product of the sample and the reagent is calorimetrically analyzed in the calorimetric autoanalyzer to determine the amount of nitrate in the sample. For nitrate analysis, the colorimetric autoanalyzer calorimetrically analyzes the sample at a wavelength between approximately 520 and 550 nanometers. For optimal nitrate analysis the colorimetric autoanalyzer calorimetrically analyzes the sample at a wavelength of 546 nanometers.

The hydrochloric acid is typically concentrated hydrochloric acid, typically approximately 0.5 molar hydrochloric acid. In one embodiment, the reagent composition of the invention comprises from about 3 to about 7% by weight concentrated hydrochloric acid, from about 0.09 to about 0.5% by weight vanadium trichloride, from about 0.1 to about 0.2% by weight sulfanilamide, from about 0.0049 to about 0.05% by weight N-1-naphthylethylenediamine, and from about 900 to about 1000 milliliters of deionized water. In one embodiment, an especially preferred reagent composition comprises approximately 41.25 milliliters of concentrated hydrochloric acid, 2.5 grams of vanadium trichloride, 1.0 gram sulfanilamide, 0.05 gram N-1-naphthylethylenediamine and between approximately 900 and 1000 milliliters of deionized water. If there are undissolved particles present in the reagent, the reagent is filtered through a less than 0.45 micron syringe filter.

Typically, the sample is allowed to react with the reagent for at least five minutes. More typically, the sample is allowed to react with the reagent for approximately fifteen minutes. The sample may react with the reagent for approximately 1105 seconds or up to ten hours.

The amounts of sample and reagent mixed to react within the calorimetric autoanalyzer is optimized to allow for accurate nitrate determination, and can be anywhere from approximately 1 part sample mixed with 150 parts reagent to 1 part reagent mixed with 150 parts sample. For example, typically, if the nitrate concentration in the sample is between approximately zero and fifty milligrams per liter, 1 part sample is mixed with approximately 94 parts reagent, such as approximately 10 microliters of sample mixed with approximately 940 microliters of reagent. Typically, if the nitrate concentration in the sample is between approximately zero and ten milligrams per liter, 1 part sample is mixed with approximately 18 parts reagent, such as approximately 50 microliters of sample mixed with approximately 900 microliters of reagent. Possibly, 1 part sample is mixed with approximately 8.5 parts reagent, such as approximately 100 microliters of sample mixed with approximately 850 microliters of reagent. Typically, if the nitrate concentration in the sample is between approximately zero and two milligrams per liter, 1 part sample is mixed with approximately 3.22 parts reagent, such as approximately 225 microliters of sample mixed with approximately 725 microliters of reagent. Typically, if the nitrate concentration in the sample is between approximately 0 and 0.2 milligrams per liter, 1 part reagent is mixed with approximately 3.22 parts sample, such as approximately 725 microliters of sample mixed with approximately 225 microliters of reagent.

In another aspect of the invention, a reagent for determining nitrate concentration comprising approximately 41.25 milliliters of concentrated hydrochloric acid, 1 gram of vanadium trichloride, 0.4 gram sulfanilamide, 0.02 gram N-1-naphthylethylenediamine and between approximately 900 and 1000 milliliters of deionized water.

### BRIEF DESCRIPTION OF THE FIGURES

The accompanying drawings form part of the specification and like numerals are employed to designate like parts throughout the same.

FIG. 1 is a top plan schematic view of an embodiment of the colorimetric autoanalyzer in accordance with the invention.

FIG. 2 is a front elevation schematic view of an embodiment of the calorimetric autoanalyzer in accordance with the invention.

### DETAILED DESCRIPTION OF THE INVENTION

While this invention is susceptible of embodiment in many different forms, there is shown in the drawings and described in detail herein, several specific embodiments with the understanding that the present disclosure is to be considered as exemplifications of the principles of the invention and is not intended to limit the invention to the embodiments illustrated.

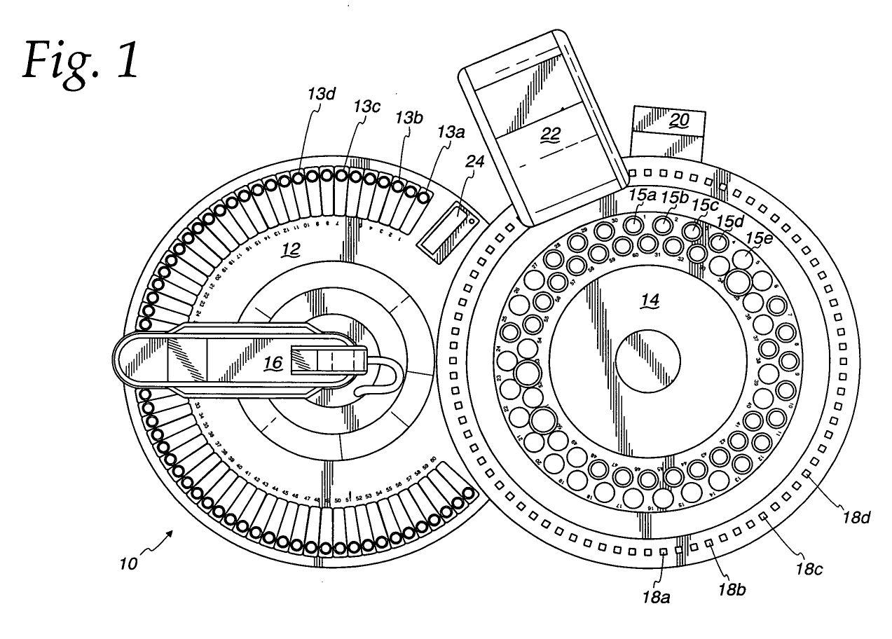

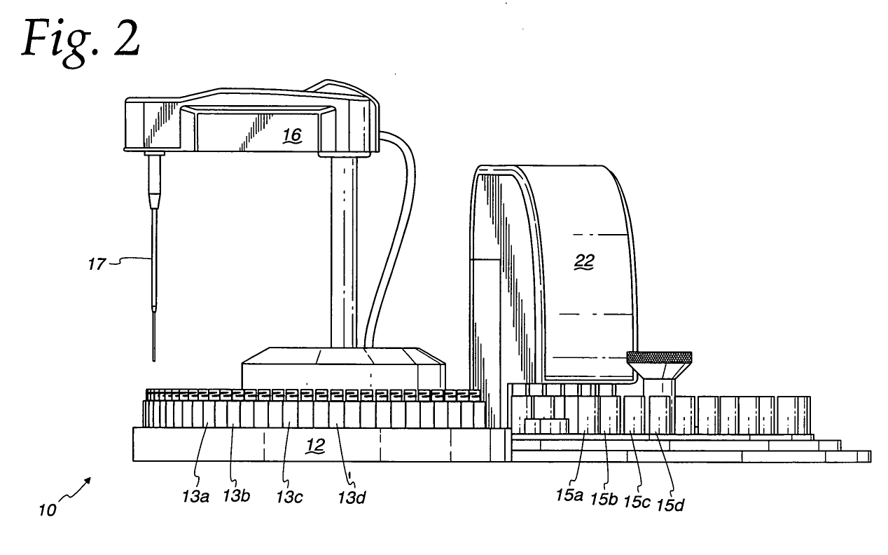

Referring to FIG. 1 and FIG. 2, a schematic of a colorimetric autoanalyzer 10 is provided. Colorimetric autoanalyzer 10 comprises a reagent carousel 12 with a plurality of reagent positions comprising reagent containers, representative ones being identified as reagent containers 13a-d, and a sample carousel 14 with a plurality of sample containers 15a-e. Several more reagent containers 13 and sample containers 15 are illustrated in FIG. 1 and FIG. 2. Typically, there are approximately sixty sample positions and sixty sample containers. Colorimetric autoanalyzer 10 may contain between approximately fifteen and seventy reagent containers. Typically, there are between approximately eighteen and thirty-six reagent containers. A sample dispensing arm 16 transfers a sample via syringe 17 from sample containers, representative ones being identified as sample containers 15a-e, to a reaction compartment 18a-d, and transfers reagent from reagent containers 13a-d to reaction compartment 18a-d. Several more reaction compartments 18 are illustrated on FIG. 1. Colorimetric autoanalyzer 10 comprises a plurality of reaction compartments. Colorimetric autoanalyzer 10 further comprises spectrophotometer 20 for analyzing the reaction product of the reaction between the sample and the reagent. Typically, reaction compartments 18a-d are cuvettes for spectrophotometric analysis of the reaction product in spectrophotometer 20. Colorimetric autoanalyzer 10 further comprises reaction compartment wash station 22 and probe wash station 24. Colorimetric autoanalyzer 10 is connected to and controlled by a computer via a serial link. Colorimetric autoanalyzers are found within the art, for example, EasyChem Pro manufactured by Systea.

In one aspect of the invention, a colorimetric autoanalyzer for determining nitrate concentration is provided. The calorimetric autoanalyzer comprises a plurality of sample positions for holding sample containers; a plurality of reagent containers for holding a reagent comprising effective amounts of hydrochloric acid, vanadium trichloride, sulfanilamide and N-1-naphthylethylenediamine for nitrate analysis; a plurality of sample positions; at least one dispensing arm to automatically transfer sample from the sample containers and reagent from the reagent containers to a reaction compartment; and a spectrophotometer for analyzing nitrate concentration, wherein the colorimetric autoanalyzer provides substantially real-time monitoring of sample results.

The autoanalyzer may comprise from approximately 1 to approximately 100 sample positions. Typically, the autoanalyzer comprises approximately 60 sample positions. Typically, the autoanalyzer comprises from approximately 18 to approximately 35 reagent containers. The autoanalyzer may have one sample dispensing arm. Typically, the autoanalyzer has at least two dispensing arms, a sample dispensing arm and a reagent dispensing arm. The dispensing arms are particular to dispensing sample or dispensing reagent only in order to increase throughput and minimize cross contamination. The sample dispensing arm transfers the sample from the sample containers into the reaction compartment. The reagent dispensing arm transfers the reagent from the reagent containers to the reaction compartment and allows the mixture to react within the reaction compartment. Alternatively, the autoanalyzer may have one dispensing arm that dispenses both sample and reagent.

A method of determining nitrate concentration in a colorimetric autoanalyzer is provided. A colorimetric autoanalyzer is provided, in which at least one sample containing nitrate is added to a sample container in the autoanalyzer and at least one reagent container in the autoanalyzer is filled with a reagent comprising effective amounts of hydrochloric acid, vanadium trichloride, sulfanilamide and N-1-naphthylethylenediamine for nitrate analysis. An amount of sample is transferred from the sample container to the reaction compartment by a sample dispensing arm. An amount of reagent is transferred from the reagent container to the reaction compartment by a reagent dispensing arm. The sample dispensing arm and reagent dispensing arm take between about 6 and 60 seconds to sample, dispense, and re-wash the dispensing arm. The dispensing arms are rewashed to prevent contamination of samples or reagents from one sample analysis to the next analysis, allowing for continuous sample processing and analysis. Typically, the sample dispensing arm and reagent dispensing arm take a maximum of about 7 seconds to sample, dispense, and re-wash the dispensing arm.

In order to achieve very high accuracy and reproducibility in drawing and dispensing sample and reagent, typically, the sample dispensing arm and the reagent dispensing arm are equipped with a high-precision diluter. The diluters are typically advanced micro-metering pumps with an acrylic head and ceramic plunger driven by a stepping motor. The diluters typically have a resolution of 0.193 microliter per pulse. The sample dispensing arm and the reagent dispensing arm typically dispense up to 500 microliters per each machine cycle.

The sample and the reagent react within the reaction compartment for a period of time as desired for the reaction to be essentially completed. The reagent reduces the nitrate to nitrite for spectrophotometric analysis. The optimum reaction time for a 90% or greater reduction of nitrate to nitrite using 2.5 grams per liter vanadium trichloride within the reagent is 1105 seconds. If the reaction is allowed to run for less than 15 minutes, the nitrate to nitrite reduction is typically below 90% and an accurate reading is not typically achieved by the calorimetric analysis. Typically, the reaction runs for at least 15 minutes. More typically, the components are allowed to react for 1105 seconds.

An increase in the amount of vanadium trichloride in the reaction is necessary for accurate determination of lower amounts of nitrate in the sample. The amounts of sample and reagent mixed to react within the calorimetric autoanalyzer is optimized to allow for accurate nitrate determination, and can be anywhere from approximately 1 part sample mixed with 150 parts reagent to 1 part reagent mixed with 150 parts sample.

For example, typically, if the nitrate concentration in the sample is between approximately zero and fifty milligrams per liter, 1 part sample is mixed with approximately 94 parts reagent, such as approximately 10 microliters of sample mixed with approximately 940 microliters of reagent.

Preferably, if the nitrate concentration in the sample is between approximately zero and ten milligrams per liter, 1 part sample is mixed with approximately 18 part reagent, such as approximately 50 microliters of sample mixed with approximately 900 microliters of reagent.

Preferably, if the nitrate concentration in the sample is between approximately zero and two milligrams per liter, 1 part sample is mixed with approximately 3.22 parts reagent, such as approximately 225 microliters of sample mixed with approximately 725 microliters of reagent.

Preferably, if the nitrate concentration in the sample is between approximately 0 and 0.2 milligrams per liter, 1 part reagent is mixed with approximately 3.22 parts sample, such as approximately 725 microliters of sample mixed with approximately 225 microliters of reagent.

Typically, 1 part sample is mixed with approximately 18 parts reagent, such as approximately 50 microliters of sample mixed with approximately 900 microliters of reagent. Typically, 1 part sample is mixed with approximately 8.5 parts reagent, such as approximately 100 microliters of sample mixed with approximately 850 microliters of reagent.

### EXAMPLE 1

A reagent for determining nitrate concentration is prepared comprising approximately 41 milliliters of approximately concentrated hydrochloric acid, 1.0 gram of vanadium trichloride, 1.0 gram sulfanilamide, 0.05 gram N-1-naphthylethylenediamine and approximately 900 milliliters of deionized water.

### EXAMPLE 2

A reagent for determining nitrate concentration is prepared comprising 41.25 milliliters of concentrated hydrochloric acid, 950 milliliters of deionized water, 2.5 grams of vanadium trichloride, 1.0 gram sulfanilamide and 0.05 gram N-1-naphthylethylenediamine. The reagent is filtered through a less than 0.45 micron syringe filter.

### EXAMPLE 3

Ten microliters of a sample with 0 to 50 milligrams per liter of nitrate is added to a sample container in a calorimetric autoanalyzer.

The reagent from Example 2 is added to a reagent container in a colorimetric autoanalyzer.

Ten microliters of a sample is transferred from the sample container to the reaction compartment via a sample dispensing arm.

940 microliters of a reagent is transferred from the reagent container to the reaction compartment via a sample dispensing arm.

The reaction is allowed to proceed for 1807 seconds, resulting in a 90% or greater conversion of nitrate to nitrite.

The reaction product is analyzed in a colorimetric autoanalyzer at a wavelength of 546 nanometers.

The colorimetric analyzer produced a reading of 25.899 ppm nitrate.

### EXAMPLE 4

A sample with 0 to 2 milligrams per liter of nitrate is added to a sample container in a calorimetric autoanalyzer.

A reagent for determining nitrate concentration is prepared comprising approximately 41 milliliters of concentrated hydrochloric acid, 2.5 grams of vanadium trichloride, 1.0 gram sulfanilamide, 0.05 gram N-1-naphthylethylenediamine and approximately 900 milliliters of deionized water is added to a reagent container in a calorimetric autoanalyzer.

225 microliters of a sample is transferred from the sample container to the reaction compartment via a sample dispensing arm.

725 microliters of a reagent is transferred from the reagent container to the reaction compartment via a sample dispensing arm.

The reaction is allowed to proceed for 1105 seconds, resulting in a 90% or greater conversion of nitrate to nitrite.

The reaction product is analyzed in a calorimetric autoanalyzer at a wavelength of 546 nanometers.

The calorimetric analyzer produced a reading of 1.567 ppm nitrate.

### EXAMPLE 5

A sample with 0 to 0.2 milligrams per liter of nitrate is added to a sample container in a calorimetric autoanalyzer.

A reagent for determining nitrate concentration is prepared comprising approximately 41 milliliters of concentrated hydrochloric acid, 4.0 grams of vanadium trichloride, 1.0 gram sulfanilamide, 0.05 grams N-1-naphthylethylenediamine and approximately 900 milliliters of deionized water is added to a reagent container in a calorimetric autoanalyzer.

725 microliters of a sample is transferred from the sample container to the reaction compartment via a sample dispensing arm.

225 microliters of a reagent is transferred from the reagent container to the reaction compartment via a sample dispensing arm.

The reaction is allowed to proceed for 1105 seconds, resulting in a 90% or greater conversion of nitrate to nitrite.

The reaction product is analyzed in a colorimetric autoanalyzer at a wavelength of 546 nanometers.

The calorimetric analyzer produced a reading of 0.142 ppm nitrate.

### EXAMPLE 6

A sample with 0 to 10 milligrams per liter of nitrate is added to a sample container in a calorimetric autoanalyzer.

The reagent from Example 4 is added to a reagent container in a colorimetric autoanalyzer.

50 microliters of a sample is transferred from the sample container to the reaction compartment via a sample dispensing arm.

900 microliters of a reagent is transferred from the reagent container to the reaction compartment via a sample dispensing arm.

The reaction is allowed to proceed for 1105 seconds, resulting in a 90% or greater conversion of nitrate to nitrite.

The reaction product is analyzed in a colorimetric autoanalyzer at a wavelength of 546 nanometers.

The calorimetric analyzer produced a reading of 8.295 ppm nitrate.

While the invention has been described with respect to certain preferred embodiments, as will be appreciated by those skilled in the art, it is to be understood that the invention is capable of numerous changes, modifications and rearrangements, and such changes, modifications and rearrangements are intended to be covered by the following claims.

1. A calorimetric autoanalyzer for determining nitrate concentration comprising:
a plurality of sample positions for holding sample containers;
a plurality of reagent containers for holding a reagent comprising effective amounts of hydrochloric acid, vanadium trichloride, sulfanilamide and N-1-naphthylethylenediamine for nitrate analysis; a plurality of sample positions;
at least one sample dispensing arm to automatically transfer sample from the sample containers to a reaction compartment; and
a spectrophotometer for analyzing nitrate concentration, wherein the colorimetric autoanalyzer provides substantially real-time monitoring of sample results.

2. The colorimetric autoanalyzer of claim 1 further comprising at least one reagent dispensing arm to automatically transfer reagent from the reagent containers to the reaction compartment.

3. The calorimetric autoanalyzer of claim 1 wherein said autoanalyzer is controlled by an external computer through a bi-directional communication link.

4. The calorimetric autoanalyzer of claim 1 wherein said autoanalyzer provides substantially real-time monitoring of sample status.

5. The calorimetric autoanalyzer of claim 1 wherein said autoanalyzer provides substantially real-time monitoring of reagent volumes and system solutions

6. The colorimetric autoanalyzer of claim 5 wherein said autoanalyzer provides substantially real-time monitoring of and waste levels.

7. A method of determining nitrate concentration using an automated colorimetric autoanalyzer comprising:
providing a calorimetric autoanalyzer;
adding at least one sample containing nitrate to a sample container in the autoanalyzer;
adding a reagent to a reagent container in the autoanalyzer, said reagent comprising effective amounts of hydrochloric acid, vanadium trichloride, sulfanilamide and N-1-naphthylethylenediamine for nitrate analysis;
mixing the sample and the reagent in the autoanalyzer;
thereafter, allowing the sample to react with the reagent sufficiently to permit determination of the amount of nitrate in the sample; and
calorimetrically analyzing the sample to determine the amount of nitrate in the sample.

8. The method of determining nitrate concentration of claim 7 wherein said hydrochloric acid is approximately 0.5 molar hydrochloric acid.

9. The method of determining nitrate concentration of claim 7 wherein the reagent comprises approximately 41.25 milliliters of approximately concentrated hydrochloric acid, 2.5 grams of vanadium trichloride, 1.0 gram sulfanilamide, 0.05 gram N-1-naphthylethylenediamine and between approximately 900 and 1000 milliliters of deionized water.

10. The method of determining nitrate concentration of claim 7 wherein the sample reacts with the reagent for at least between 5 and 15 minutes.

11. The method of determining nitrate concentration of claim 7 wherein the sample reacts with the reagent for at least 1105 seconds.

12. The method of determining nitrate concentration of claim 7 wherein approximately 10 microliters of the sample containing nitrate and approximately 940 microliters of the reagent are allowed to react in the autoanalyzer.

13. The method of determining nitrate concentration of claim 9 wherein approximately 225 microliters of the sample containing nitrate and approximately 725 microliters of the reagent are allowed to react in the autoanalyzer.

14. The method of determining nitrate concentration of claim 9 wherein approximately 50 microliters of the sample containing nitrate and approximately 900 microliters of the reagent are allowed to react in the autoanalyzer.

15. The method of determining nitrate concentration of claim 9 wherein approximately 10 microliters of the sample containing nitrate and approximately 940 microliters of the reagent are allowed to react in the autoanalyzer.

16. The method of determining nitrate concentration of claim 9 wherein the reagent comprises approximately 4.0 grams of vanadium trichloride and wherein approximately 725 microliters of the sample containing nitrate and approximately 225 microliters of the reagent are allowed to react in the autoanalyzer.

17. The method of determining nitrate concentration of claim 9 wherein approximately 100 microliters of the sample containing nitrate and approximately 850 microliters of the reagent are allowed to react in the autoanalyzer.

18. The method of determining nitrate concentration of claim 7 wherein the calorimetric autoanalyzer calorimetrically analyzes the sample at a wavelength between approximately 520 and 550 nanometers.

19. The method of determining nitrate concentration of claim 18 wherein the colorimetric autoanalyzer calorimetrically analyzes the sample at a wavelength of approximately 546 nanometers.

20. A reagent for determining nitrate concentration comprising approximately 41.25 milliliters of concentrated hydrochloric acid, 1 gram of vanadium trichloride, 1.0 gram sulfanilamide, 0.05 gram N-1-naphthylethylenediamine and between approximately 900 and 1000 milliliters of deionized water.

21. A reagent for determining nitrate concentration comprising approximately 41 milliliters of concentrated hydrochloric acid, 4.0 grams of vanadium trichloride, 1.0 gram sulfanilamide, 0.05 gram N-1-naphthylethylenediamine and between approximately 900 and 1000 milliliters of deionized water.
